(19) United States
(12) Patent Application Publication
(10) Pub. No.: US 2011/0105978 A1
(43) Pub. Date: May 5, 2011

(54) RESILIENT PROTECTIVE WRAP

(76) Inventor: I-Yung Hou, Tainan County (TW)

(21) Appl. No.: 12/727,373

(22) Filed: Mar. 19, 2010

(30) Foreign Application Priority Data
Oct. 29, 2009 (TW) 098219984

(51) Int. Cl. A61F 5/00; B32B 3/26

(52) U.S. Cl. 602/75; 428/220

(57) ABSTRACT

A resilient protective wrap usable to wrap a body part of a user for protection against injury includes at least one resilient fabric layer that is stretchable at least in one direction, and an open cell water-based polyurethane foam laminated on the resilient fabric layer and made from a material including an aqueous dispersion of polyurethane. The polyurethane foam has a thickness of at least 1 mm, a largest elongation ranging from 200%-450%, a modulus of elasticity ranging from 2 to 7 kgf/cm², a tensile strength of at least 4 kgf/cm², and a density ranging from 180-600 g/l.

### CROSS-REFERENCE TO RELATED APPLICATION

This application claims priority of Taiwanese Application No. 098219984, filed on Oct. 29, 2009.

### BACKGROUND OF THE INVENTION

1. Field of the Invention

This invention relates to a resilient protective wrap, more particularly to a resilient protective wrap to wrap and support a body part of an individual.

2. Description of the Related Art

Resilient protective wraps, such as wrist bands, hand braces, waist bands, ankle supports, etc., may be used to wrap a body part of an individual during therapy, body exercise, sports, and other activities in order to support bones, and especially to protect joints. A typical resilient protective wrap is made from a multi-layered textile material that includes two stretchable fabric layers sandwiching a layer of rubber, such as neoprene or chloroprene rubber. Such rubber materials are highly elastic to provide stretchability, exhibit a good restoring force to provide sufficient compression fit to the body part of the user, and possess high tensile strength to impart high durability. However, there are disadvantages in that the rubber materials can pollute the environment and can cause discomfort to the user due to their poor heat-dissipating and air-circulating ability.

Water-based polyurethane is solvent-free and environmentally friendly. Open cell foams made from an aqueous dispersion of polyurethane are breathable and have been used commonly in the manufacture of synthetic leather and other textile products which do not require high elasticity and a good restoring force. Currently, open cell foams made from an aqueous polyurethane dispersion are usually used as a coating having a small thickness that is less than 1 mm.

### SUMMARY OF THE INVENTION

Therefore, the object of the present invention is to provide a resilient protective wrap that is environmentally friendly and that has good heat-dissipating and air-circulating properties.

According to one aspect of this invention, a resilient protective wrap usable to wrap a body part of a user for protection against injury comprises at least one resilient fabric layer that is stretchable at least in one direction, an open cell water-based polyurethane foam laminated on the resilient fabric layer and made from a material including an aqueous dispersion of polyurethane. The polyurethane foam has a thickness of at least 1 mm, a largest elongation ranging from 200%-450%, a modulus of elasticity ranging from 2 to 7 kgf/cm², a tensile strength of at least 4 kgf/cm², and a density ranging from 180-600 g/l.

According to another aspect of this invention, an open cell polyurethane foam is made from a material that includes an aqueous dispersion of water-based polyurethane, and has a thickness of at least 1 mm, a largest elongation ranging from 200%-450%, a modulus of elasticity ranging from 2 to 7 kgf/cm², a tensile strength of at least 4 kgf/cm², and a density ranging from 180-600 g/l.

### BRIEF DESCRIPTION OF THE DRAWINGS

Other features and advantages of the present invention will become apparent in the following detailed description of the preferred embodiments of the invention, with reference to the accompanying drawings, in which:

FIG. 1 is a perspective view showing a resilient protective wrap according to the present invention embodied as a knee brace;

FIG. 2 is a sectional view taken along line 2-2 of FIG. 1; and

FIG. 3 is a perspective view showing another resilient protective wrap according to the present invention embodied as a wrist brace.

### DETAILED DESCRIPTION OF THE PREFERRED EMBODIMENTS

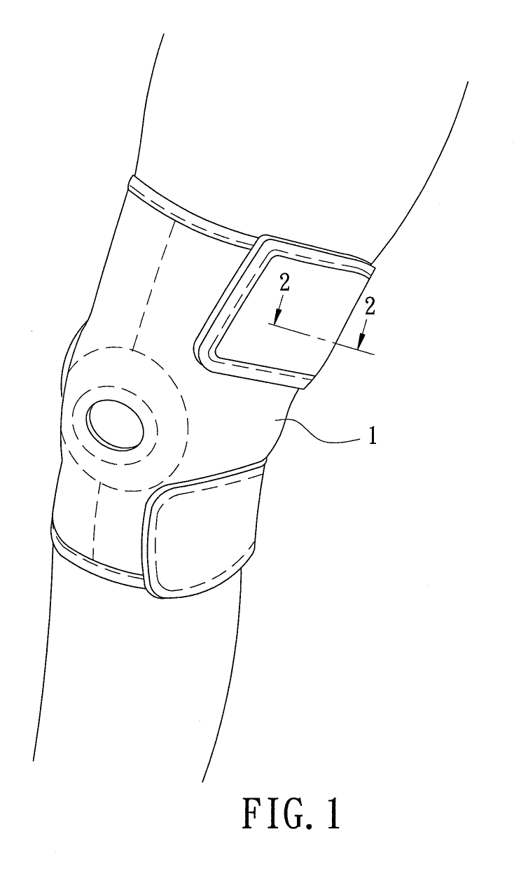

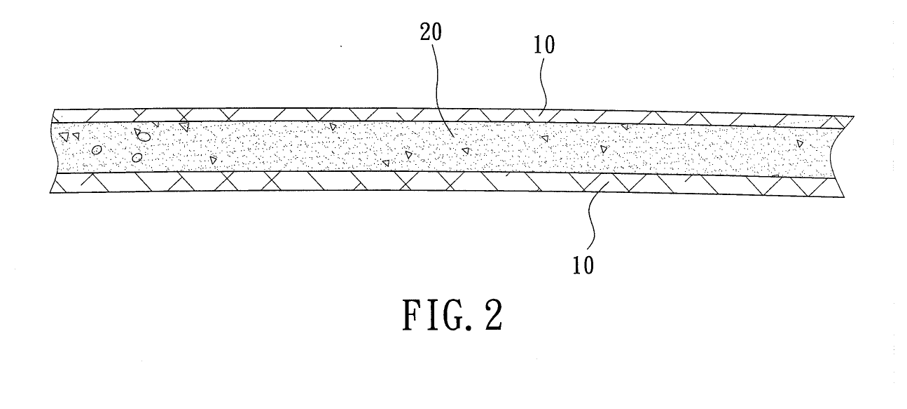

Referring to FIGS. 1 and 2, there is shown a resilient protective wrap according to the present invention that is embodied as a knee brace 1. The knee brace 1 includes a multi-layered textile structure that includes top and bottom fabric layers 10, and an open cell polyurethane foam 20 laminated between the fabric layers 10.

The top and bottom fabric layers 10 are made from a stretchable fabric material, such as a woven or knitted fabric. The fabric layers 10 may have a unidirectional stretch fabric that is stretchable along a longitudinal direction of the knee brace 1, or a bidirectional stretch fabric that is stretchable in either a longitudinal or transverse direction.

The open cell polyurethane foam 20 is made from a material including an aqueous dispersion of water-based polyurethane (PU). The aqueous polyurethane dispersion used in the present invention may be prepared from a polyol component and a crosslinking agent. The polyol component may include a polyether polyol or a polyester polyol. The crosslinking agent may include an amine resin or an isocyanate compound. Any polyol and crosslinking agent suitable for forming an aqueous polyurethane dispersion may be used in the present invention.

In addition to the aqueous polyurethane dispersion, the material of the open cell polyurethane foam 20 further includes other ingredients, such as a foaming agent, a foam stabilizer, a thickener, a pigment, a filler or extender, etc. The foaming agent, foam stabilizer, thickener, pigment, and filler may be compounds that are used conventionally in preparing a foam-forming polyurethane composition. Examples of the fillers or extenders used in the present invention include wood flour, calcium carbonate, mica, etc. Preferably, the fillers or extenders are nano particle materials.

The water-based polyurethane foam 20 may be made in a conventional manner. In an embodiment, the ingredients of the material are mixed and foamed using a foaming mixer. The resulting foamed material may be laminated with the fabric layers 10 through a conventional lamination method. For instance, the foamed material may be spread onto a surface of one of the fabric layers 10 that is advanced by a conveyor, and may be heated for removal of water from the foamed material while being advanced on the conveyor. As the water is removed gradually, the foamed material becomes a semisolid, and another fabric layer 10 may be placed on the surface of the foamed material. Continued heating of the foamed material will cause the material to become hardened completely. After the foamed material is cooled, it is adhered to both of the fabric layers 10 without using any adhesive.

The thickness of the foamed material spread onto the fabric layer 10 may be controlled using a doctor blade. Preferably, the polyurethane foam 20 has a thickness of at least 1 mm, more preferably ranging from 1-6 mm, and most preferably 2-5 mm.

The degree of foaming or the density of the polyurethane foam 20 may be adjusted by controlling the foaming mixer or the duration time for foaming the material. Generally, when the density of the polyurethane foam 20 is decreased, the elongation thereof increases but the tensile strength and the modulus of elasticity thereof will decrease. When the density of the polyurethane foam 20 is increased, the tensile strength and the modulus of elasticity increase, but the elongation will decrease. Preferably, the density of the polyurethane foam 20 is controlled to range from 180-600 g/l, more preferably 200-450 g/l.

In order to enable the resilient protective wrap to provide good tightness and sufficient compression to the user when the resilient protective wrap is worn by the user, the polyurethane foam 20 is provided with a modulus of elasticity ranging from 2 to 7 kgf/cm², preferably 3-6 kgf/cm², and more preferably 4.0-5.5 kgf/cm². If the modulus of elasticity of the polyurethane foam 20 is lower than 2 kgf/cm², the resilient protective wrap will be too slack to provide good tightness and sufficient wrapping pressure to the user. If the modulus of elasticity is higher than 7 kgf/cm², the resilient protective wrap will be too tight and will cause discomfort to the user.

The largest elongation of the polyurethane foam 20 preferably ranges from 200% to 450%, more preferably from 250% to 350%. If the largest elongation is lower than 200%, the polyurethane foam 20 cannot have sufficient stretchability so that the resilient protective wrap will be overly tight. If the largest elongation is higher than 450%, the stretchability of the polyurethane foam 20 will be overly high so that the resilient protective wrap cannot provide sufficient tightness and therefore insufficiently supports the particular joint of the user around which the resilient protective wrap is provided.

In order to provide the polyurethane foam 20 with sufficient robustness, the tensile strength thereof is preferably at least 4 kgf/cm², and more preferably at least 6 kgf/cm².

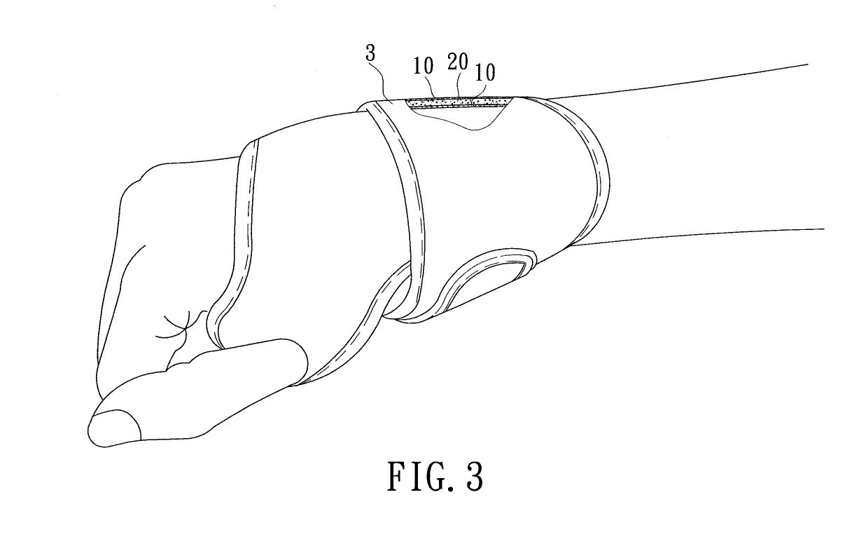

Referring to FIG. 3, there is shown another resilient protective wrap according to the present invention that is embodied as a wrist brace 3. The wrist brace 3 also has a multi-layered textile structure including the fabric layers 10 and the polyurethane foam 20.

Specific examples of the open cell resilient foam 20 will be further illustrated below. It should be understood that the Examples are for illustrative purposes only and are not to be construed as limiting the scope of the present invention in any manner whatsoever.

### EXAMPLES

### Preparation of the Material for the Open Cell Resilient Foam

### Formulation 1

100 parts by wt of polyether polyol (40 parts of Impranil LP RSC 1380 and 60 parts of Impranil DLU from Bayer, particle size 200-300 nm, solid content 60%) was mixed with the following ingredients:

2-8 parts by wt of a crosslinking agent (TDI, Desmodur N3900 from Bayer) based on a total solid content of the polyol;

2-6 parts by wt of a foaming agent (succinamate, Stockal SR from Bozzetto Group) based on a total solid content of the polyol;

2-8 parts by wt of a foam stabilizer (Stockal STA from Bozzetto Group) based on a total solid content of the polyol;

1-5 parts by wt of a thickener (Borchi Gel ALA from OMG Borchers GmbH) based on a total solid content of the polyol;

3-6 parts by wt of a white pigment based on a total solid content of the polyol; and

1-4 parts by wt of a filler or extender.

### Formulation 2

100 parts by wt of polyester type polyol (70 parts of Impranil LP RSC 1554 and 30 parts of Impranil LP RSC 1537 from Bayer, particle size 200-300 nm, solid content 60%) was mixed with the following ingredients:

2-8 parts by wt of a crosslinking agent (TDI Desmodur N3900 from Bayer) based on a total solid content of the polyol;

5-10 parts by wt of a foaming agent (Dicrylan FLN from Huntsman) based on a total solid content of the polyol;

1-5 parts by wt of a thickener (Mirox Am from Bozzetto Group) based on a total solid content of the polyol;

3-6 parts by wt of a white pigment based on a total solid content of the polyol; and

1-4 parts by wt of a filler or extender.

### Examples 1-12

Examples 1-12 were prepared using formulation (1) and using different fillers listed in Tables 1-3. The mica used in Example 1 and Examples 5-12 had a particle size of 5-20 nm.

### Fabrication of the Polyurethane Foam

The ingredients of each of Examples 1-12 were mixed and foamed in a mixer. The resulting foamed mixture of each example was spread on a release substrate to form a foam layer and was heated gradually to a temperature of about 140° C. in order to completely remove water from the foamed mixture. The thickness of the foam layer spread on the release substrate was controlled by using a doctor blade.

Samples of Examples 1-12 were subjected to tests for tensile strength, elongation, and modulus of elasticity according to standard method CNS-3553. The properties of the samples are shown in Tables 1 to 3.

TABLE 1

| | | | | | | Modulus |
|---|---|---|---|---|---|---|
| | | | | Tensile | Largest | of |
| | | Thick- | | Strength | Elon- | Elasticity |
| Exam- | | ness | Density | (Kgf/ | gation | (at 200%) |
| ple | Filler | (mm) | (g/l) | cm²) | (%) | (Kgf/cm²) |
| 1 | Mica 1% | 3.0 | 230 | 8.184 | 297.628 | 5.22 |
| 2 | CaCO₃ 1% | 3.0 | 230 | 7.478 | 318.878 | 4.36 |
| 3 | Wood | 3.0 | 230 | 6.721 | 262.502 | 5.15 |
| | flour 1% | | | | | |
| 4 | No filler | 3.0 | 230 | 7.923 | 311.503 | 4.91 |

TABLE 2

| | | | | | | Modulus |
|---|---|---|---|---|---|---|
| | | | | Tensile | Largest | of |
| | | Thick- | | Strength | Elon- | Elasticity |
| Exam- | | ness | Density | ((Kgf/ | gation | (at 200%) |
| ple | Filler | (mm) | (g/l) | cm²) | (%) | (Kgf/cm²) |
| 5 | Mica | 2.3 | 159 | 4.648 | 431.477 | 2.14 |
| | 1% | | | | | |
| 6 | Mica | 2.3 | 306 | 7.289 | 366.047 | 3.51 |
| | 1% | | | | | |
| 7 | Mica | 2.3 | 459 | 10.08 | 339.798 | 5.00 |
| | 1% | | | | | |
| 8 | Mica | 2.3 | 609 | 10.932 | 269.625 | 6.376 |
| | 1% | | | | | |

TABLE 3

| | | | | | | Modulus |
|---|---|---|---|---|---|---|
| | | | | Tensile | Largest | of |
| | | Thick- | | Strength | Elon- | Elasticity |
| Exam- | | ness | Density | ((Kgf/ | gation | (at 200%) |
| ple | Filler | (mm) | (g/l) | cm²) | (%) | (Kgf/cm²) |
| 9 | Mica | 2.3 | 240 | 5.147 | 345.755 | 2.69 |
| | 2% | | | | | |
| 10 | Mica | 2.3 | 238 | 4.784 | 342.003 | 2.80 |
| | 3% | | | | | |
| 11 | Mica | 2.3 | 236 | 4.421 | 333.294 | 2.69 |
| | 5% | | | | | |
| 12 | Mica | 2.3 | 236 | 4.189 | 313.211 | 3.03 |
| | 7% | | | | | |

The results of Table 1 show that the tensile strength and the modulus of elasticity of Example 1 is higher than that of Examples 2-4. It was observed that Example 1 provides good tightness and compression without causing discomfort to the user.

The results of Table 2 show that, when the density of the polyurethane foam is increased, the tensile strength and the modulus of elasticity increase, and the elongation decreases.

The results of Table 3 show that the amount of mica used in the polyurethane foam can affect the properties of the polyurethane foam.

In sum, the resilient protective wrap according to the present invention, which is made including the open cell polyurethane foam, provides good heat dissipation and air circulation effects as compared to the conventional wrist brace or ankle support which is made from non-breathable rubber. Additionally, as the material used in making the polyurethane foam is solvent-free, it is environmentally friendly.

While the present invention has been described in connection with what are considered the most practical and preferred embodiments, it is understood that this invention is not limited to the disclosed embodiments but is intended to cover various arrangements included within the spirit and scope of the broadest interpretations and equivalent arrangements.

I claim:

1. A resilient protective wrap usable to wrap a body part of an individual for protection against injury, comprising:
at least one resilient fabric layer that is stretchable at least in one direction; and
an open cell water-based polyurethane foam laminated on the resilient fabric layer and made from a material including an aqueous dispersion of polyurethane;
wherein the polyurethane foam has a thickness of at least 1 mm, a largest elongation ranging from 200%-450%, a modulus of elasticity ranging from 2 to 7 kgf/cm², a tensile strength of at least 4 kgf/cm², and a density ranging from 180-600 g/l.

2. The resilient protective wrap of claim 1, wherein the largest elongation ranges from 250-350%.

3. The resilient protective wrap of claim 1, wherein the tensile strength is at least 6 kgf/cm².

4. The resilient protective wrap of claim 1, wherein the modulus of elasticity ranges from 3-6 kgf/cm².

5. The resilient protective wrap of claim 1, wherein the density ranges from 200-450 g/l.

6. The resilient protective wrap of claim 1, wherein the thickness of the polyurethane foam is 1-6 mm.

7. The resilient protective wrap of claim 1, wherein the material of the polyurethane foam further includes a foaming agent, a crosslinking agent, and a filler.

8. The resilient protective wrap of claim 7, wherein the filler includes mica particles.

9. The resilient protective wrap of claim 7, wherein the filler includes nano particles.

10. The resilient protective wrap of claim 1, wherein the resilient fabric layer is a unidirectional stretch fabric that is stretchable in a longitudinal direction of the resilient protective wrap.

11. The resilient protective wrap of claim wherein the resilient fabric layer is a bidirectional stretch fabric that is stretchable in longitudinal and transverse directions of the resilient protective wrap.

12. An open cell polyurethane foam made from a material that includes an aqueous dispersion of water-based polyurethane, and that has a thickness of at least 1 mm, a largest elongation ranging from 200-450%, a modulus of elasticity ranging from 2 to 7 kgf/cm², a tensile strength of at least 4 kgf/cm², and a density ranging from 180-600 g/l.

13. The open cell polyurethane foam of claim 12, wherein the material further includes a foaming agent, a crosslinking agent, and a filler.

14. The open cell polyurethane foam of claim 13, wherein the filler includes mica particles.

15. The open cell polyurethane foam of claim 13, wherein the filler includes nano particles.
